(19) United States
(12) Patent Application Publication
(10) Pub. No.: US 2024/0307047 A1
(43) Pub. Date: Sep. 19, 2024

(54) TISSUE RETRACTOR WITH AN INTEGRATED OPTICAL SENSOR FOR PRESSURE MEASUREMENT

(71) Applicant: David Wayne HOLDSWORTH, London (CA)

(72) Inventors: David Wayne HOLDSWORTH, London (CA); Lee David SIKSTROM, London (CA)

(21) Appl. No.: 18/602,288

(22) Filed: Mar. 12, 2024

Related U.S. Application Data
(60) Provisional application No. 63/452,411, filed on Mar. 15, 2023.

(51) Int. Cl. A61B 17/02; G16H 40/63; A61B 17/00; A61B 90/00; A61B 90/30

(52) U.S. Cl. CPC A61B 17/02; G16H 40/63; A61B 2017/00057; A61B 2017/00084; A61B 2017/00119; A61B 2090/065; A61B 2090/306

(57) ABSTRACT

A system and method simultaneously measure retraction pressure and oximetry during surgical procedures. The system involves an optical sensor module that is integrated within a retractor. Real-time optical data from the sensor module is analyzed, via machine learning or other algorithms, to determine the pressure applied between the retractor and tissue. This real-time continuous monitoring of pressure is coupled with simultaneous reporting of perfusion-related metrics at the site, providing warnings to surgeons when tissue viability is being compromised by prolonged reduction in perfusion due to retraction.

### CROSS-REFERENCE TO RELATED APPLICATION

This application claims the benefit of U.S. Provisional patent application U.S. Ser. No. 63/452,411 filed Mar. 15, 2023, the entire contents of which is herein incorporated by reference.

### FIELD OF THE INVENTION

The present invention is directed to the field of monitoring tissue viability during surgical retraction, using an optical sensor module integrated within a surgical device.

### BACKGROUND ART

Every year there are approximately 13.8 million neurosurgery cases worldwide. When surgical targets are below the surface of the brain, healthy brain tissue must be moved out of the way in a process called retraction. Tissue retractors are used during surgery to provide access to surgical targets, such as tumours or vessel malformations. Continuous retraction pressure over long intervals can lead to reduced local tissue perfusion and potential tissue damage; this is especially true in brain or nerve tissue which lacks the capacity for recovery after re-perfusion. In fact, over 40% of patients who undergo retraction may suffer brain injuries from the pressure applied to the healthy brain tissue during the procedure, no matter how careful the surgeon is. Similarly, when surgery involves the retraction of tissue around nerves, or nervous tissue itself, one of the major causes of sensory disturbance is due to the reduction of the metabolic supply to the nervous tissue from mechanical trauma.

It would be desirable to provide an indication to surgeons that would warn of potential tissue damage due to retraction, so that a retractor could be released or re-positioned. There have been tools developed to measure the local pressure applied to brain and nervous tissue during retraction, but they have not been widely adopted due to their size, complexity, or other limitations.

Previous inventions have used pneumatic, piezoelectric, capacitive, strain gauge, or other such electromechanical systems to estimate the amount of force or pressure applied to tissue during the retraction process. These pressure-measuring systems add considerable bulk and size to the retractor blades. This makes it difficult to produce a small, simple, compact retractor that can measure all the parameters such as applied pressure, heart rate, and blood oxygenation of retracted tissue. Ideally, such a retractor would be compact, inexpensive, and temporally responsive (providing real time pressure measurements), as nervous tissue is very sensitive to injury during surgical procedures.

Photoplethysmography (PPG) is a technique that uses reflected or transmitted optical signals to provide physiological measurements of oxygenation and pulse rate. Contact pressure is considered to be an undesirable confounding factor that affects the quality of PPG signals when PPG is used to monitor vital signs, and the relationship between contact pressure and the properties of the reflected or transmitted optical signal is complex.

The current invention takes advantage of this complex relationship between contact pressure and reflected or transmitted light signal to deduce the applied pressure to tissue during surgical retraction. Reflected or transmitted light (at multiple wavelengths) is analysed in real time to provide a continuous estimate of applied pressure, using an ultra-miniature optical sensor module that is small enough to be embedded directly within a retractor or other surgical device.

### SUMMARY OF THE INVENTION

The invention described is a system and method where one or more light sources and optical sensors are integrated within a medical device (e.g., a tissue retractor), that presses against tissue, and the optical signals received by the sensor(s) are used to derive the pressure applied by the medical device on the tissue. The complex relationship between the optical signals and the applied pressure may be modeled through conventional regression analysis or through machine-learning algorithms.

In one embodiment, LED light sources at multiple wavelengths are embedded in the tip of the medical device and the reflected light signal is acquired as a time series by a sensitive photodiode.

A pre-determined numerical algorithm is used to predict the applied pressure, based on analysis of the time-varying optical signals, and a warning (e.g., auditory and/or visual) is provided to the surgeon if the pressure exceeds an operator-specified limit (in pressure, time, or a combination of the two).

In some embodiments, a system for measuring pressure exerted on a tissue is provided. The system comprises: a medical device that exerts pressure on the tissue when the medical device is in contact with the tissue; one or more optical sources coupled to the medical device, the one or more optical sources configured to direct an incident light signal to the tissue during operation of the medical device; one or more optical sensors coupled to the medical device, the one or more optical sensors configured to receive and measure light-intensity, as a function of time, of a reflected and/or transmitted light signal that is reflected and/or transmitted from the tissue during the operation of the medical device; a processor in electronic communication with the one or more optical sensors, the processor provided with machine-executable instructions to analyze the light-intensity as a function of the time to derive the pressure that the medical device exerts on the tissue at any given time; and, one or more output devices for reporting information about the pressure the device exerts on the tissue at any given time.

In some embodiments, at least one of the one or more optical sources emits light at one or more wavelengths. In some embodiments, at least one of the one or more optical sources emits continuous light or pulsed light. In some embodiments, at least one the one or more optical sources comprises a light emitting diode or a laser. In some embodiments, at least one of the one or more optical sensors detects light within a range of wavelengths that includes the one or more wavelengths of the emitted light.

In some embodiments, the one or more optical sources and the one or more optical sensors are integrated into a single module coupled to the medical device, for example incorporated within a body of the medical device. In some embodiments, the one or more optical sources and the one or more optical sensors are independent components coupled to the medical device, for example independently incorporated into the body of the medical device. In some embodiments, the one or more optical sources and/or the one or more optical sensors are built into the medical device. In some embodiments, the one or more optical sources and/or the one or more optical sensors are part of a flexible covering that covers the medical device and the one or more optical sources and the one or more optical sensors are nevertheless coupled to the medical device.

In some embodiments, the one or more optical sources and/or the one or more optical sensors are directly in contact with the tissue. In some embodiments, the one or more optical sources and/or the one or more optical sensors are not directly in contact with the tissue and the light signals are guided by light guides, for example fiber optic fibers or other light guides.

In some embodiments, the medical device comprises a retractor configured to retract a portion of the tissue. In some embodiments, the one or more optical sources and/or the one or more optical sensors are incorporated within a surgical robot, which may retract or resect tissue. In some embodiments, the one or more optical sources and/or the one or more optical sensors are incorporated into a skin-sensing monitor, which may be used to monitor pressure to prevent bedsores. In some embodiments, the one or more optical sources and/or the one or more optical sensors are incorporated into a prosthetic socket to provide feedback on pressure against a tissue stump.

In some embodiments, measuring the light-intensity of the reflected and/or transmitted light signal comprises digitally measuring the reflected and/or transmitted light signal received by each of the one or more optical sensors; communicating the light-intensity measurement to the processor; storing a time series of the light-intensity measurements in an electronic memory; and, reporting the light-intensity measurements using the one or more output devices.

In some embodiments, the processor is incorporated into the medical device. In some embodiments, the processor is external to the medical device. The processor may be any suitable device for receiving electronic communication from the one or more optical sensors and for executing machine executable instructions configured to execute an analysis program. In some embodiments, the processor comprises a single-board micro-controller. The processor may be in wired or wireless communication with the one or more optical sensors. In some embodiments, the system further comprises a communication port for coupling the one or more optical sensors to the processor. In some embodiments, the communication port comprises a Universal Serial Bus (USB) port, an IEEE 1384 port, a serial port, a parallel port, a Personal Computer Memory Card International Association (PCMCIA) port, an Inter-Integrated Circuit (I2C) port, a Small Computer System Interface (SCSI) port, an optical port, a coaxial port, a Registered Jack 45 (RJ45) port and a Registered Jack 11 (RJ11) port, or a connector/connection for a mobile electronic device. In some embodiments, the processor is external to the medical device and the system further comprises a wireless protocol for the electronic communication between the one or more optical sensors and the processor. Wireless protocols include, for example, Bluetooth™. Electronic components of the system may be powered by an electrical power source, for example mains of a building, a battery, a capacitor, a solar cell, or the like. In some embodiments, the power source comprises a battery.

The system comprises one or more output devices for reporting information about the pressure the device exerts on the tissue at any given time. In some embodiments, the one or more output devices are used to report information about other parameters that may be measured or determined in connection with the operation of the medical device or the tissue. In some embodiments, the one or more output devices comprises one or more of a visual display (e.g., a monitor, a screen, a light, or the like), an auditory display (e.g., a speaker, a buzzer, or the like), a tactile stimulator, or the like.

In some embodiments, the analysis program determines the exerted pressure from the light-intensity as a function of the time using an algorithm that has been trained to correlate the light-intensity as a function of the time to the exerted pressure at any given time. In some embodiments, the analysis program reports the information about the pressure using the one or more output devices. In some embodiments, the algorithm comprises decision trees, Kth nearest neighbours, neural networks, support vectors or XGBoost™. Details of various useful algorithms useful in developing the analysis program are described in the following documents, the entire contents of all of which are herein incorporated by reference:

Abadi M, et al. TensorFlow: A system for large-scale machine learning. 12th USENIX Symposium on Operating Systems Design and Implementation. (2016) USENIX Association. 265-283.

Breiman L. Random Forests. Machine Learning, 45, 5-32, 2001. Kluwer Academic Publishers.

Elith J, et al. A working guide to boosted regression trees. Journal of Animal Ecology. 2008, 77, 802-813.

Kingma DP, et al. ADAM: A METHOD FOR STOCHASTIC OPTIMIZATION. arXiv:1412.6980v9 [cs.LG] 30 Jan. 2017. Published as a conference paper at ICLR 2015.

Liaw A, et al. Classification and Regression by randomForest. R News. Vol. 2/3, December 2002, ISSN 1609-3631.

Pedregosa F, et al. Scikit-learn: Machine Learning in Python. Journal of Machine Learning Research 12 (2011) 2825-2830.

In some embodiments, the information about the pressure comprises a warning. In some embodiments, the analysis program determines when a pre-set pressure and/or time threshold has been exceeded, and the analysis program provides the warning as part of the information reported using the one or more output devices. In some embodiments, the warning comprises a warning light, a tactile indication, a sound or any combination thereof. In some embodiments, the information comprises a report of when a specified pressure threshold is reached or a plot showing a history of the exerted pressure over time.

In some embodiments, the light-intensity of the reflected and/or transmitted light signal is used to measure vital signs of a patient. In some embodiments, the vital signs are simultaneously reported with the pressure exerted on the tissue by the medical device using the one or more output devices. In some embodiments, the vital signs comprise one or more of heart rate, blood oxygenation and blood pressure. In some embodiments, the system further comprises a temperature sensor for measuring temperature where the medical device contacts the tissue.

Thus, the system and the method can be used to simultaneously measure pressure exerted by the medical device (e.g., retraction pressure) and oximetry during surgical procedures. Real-time optical data from the one or more sensors is analyzed, via machine learning or other algorithms, to determine the pressure applied between the medical device and the tissue. This real-time continuous monitoring of pressure can be coupled with simultaneous reporting of perfusion-related metrics at a surgical site, providing warnings to surgeons when tissue viability is being compromised by prolonged reduction in perfusion due to the procedure (e.g., retraction).

### BRIEF DESCRIPTION OF THE DRAWINGS

FIG. 1: Diagram of an example retractor blade with the optical sensor mounted in the end that contacts the tissue.

FIG. 2: Details of an example embodiment of the optical sensor device showing the light sources and the light detector in a retractor blade.

FIG. 3: Side view of a retractor blade with sensor mounted.

FIG. 4: Overall data acquisition and processing pathway, illustrated as a flow chart.

FIG. 5: Illustration of one embodiment of the miniaturized optical sensor module integrated into a test platform for development of the analysis algorithm.

FIG. 6: Illustration of the mechanical set up of a device to collect simultaneous optical data and force or pressure data for training, validation, and testing of the machine-learning algorithm.

FIG. 7: Flow chart describing one example of the training process for a machine-learning algorithm, based on multi-spectral optical data and known applied tissue pressure.

FIG. 8: Illustrates the real-time performance of one machine-learning algorithm (Random Forest Regression) in estimating the applied force on the sensor based on multi-spectral reflected light signals.

FIG. 9: Illustration of the performance of one machine-learning algorithm (Random Forest Regression) in estimating the applied force on the sensor based on multi-spectral reflected light signals, over a range of applied loads.

### DETAILED DESCRIPTION OF THE INVENTION

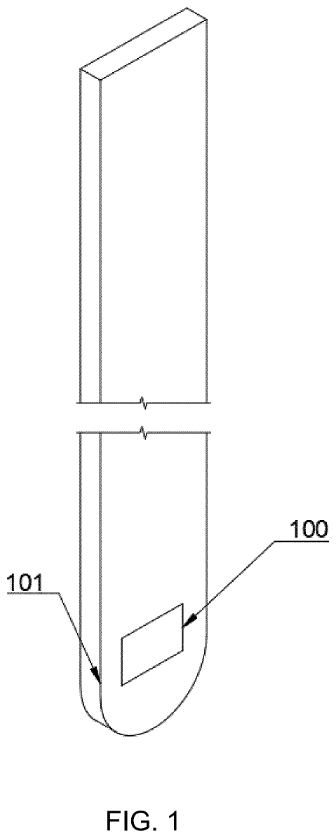

Referring to FIG. 1, one embodiment of the device is implemented as a thin, metal retractor blade 101 that contains at least one integrated optical sensor module 100 (light source and detector) in the tip.

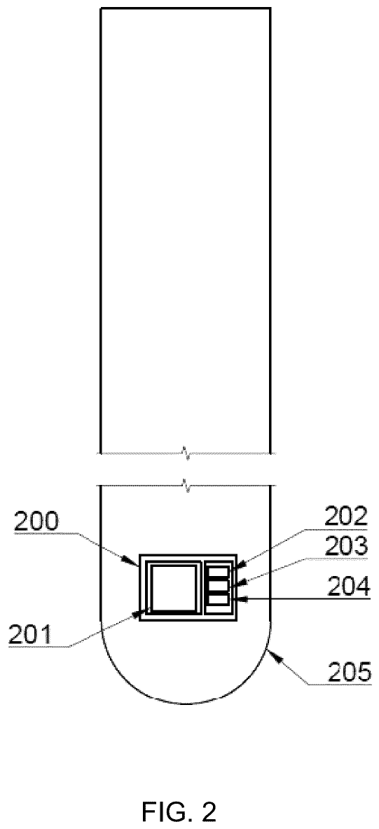

Referring to FIG. 2, integrated optical sensor module 200 incorporates a sensitive photodiode light sensor 201 combined with light sources at multiple wavelengths. In one embodiment, the light sources are light-emitting diodes (LEDs) 202, 203, 204 operating at wavelengths of 530 nm, 660 nm, and 880 nm, respectively. The integrated optical sensor module is positioned near the tip of the surgical retractor 205.

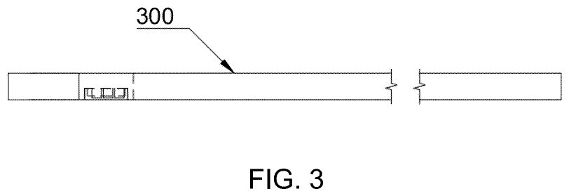

Referring to FIG. 3, integrated optical sensor module 300 is integrated within the surgical retractor such that it maintains a smooth continuous surface that can apply pressure to the tissue and simultaneously record and analyze reflected light signals at multiple wavelengths.

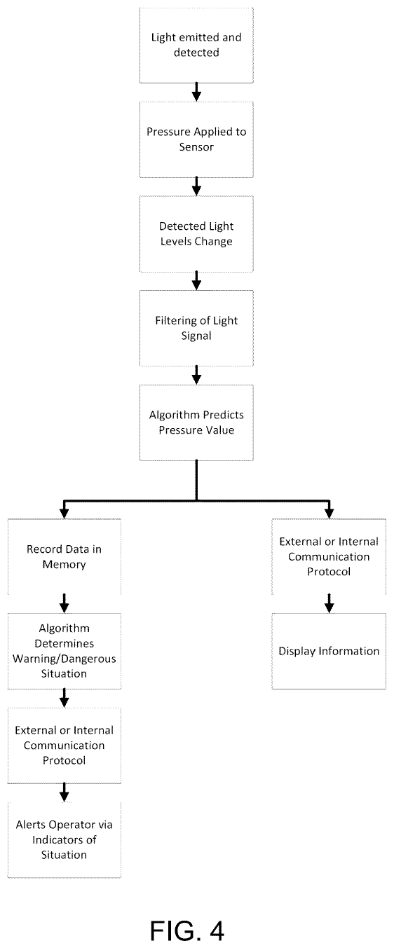

Referring to FIG. 4, the overall acquisition and data processing pathway is illustrated in a flow chart, which describes the emission and detection of light at the tip of the retractor as it applies pressure to the tissue. Light within multiple frequency bands is detected, filtered and analysed to determine the contact pressure applied to the tissue by the retractor blade using various real-time data analysis techniques, including machine learning. The optical signals could also be used to determine other biologically important signals such as heart rate, blood oxygenation, and blood pressure, simultaneously with retractor pressure. The output of the pressure measurements can be recorded, displayed or communicated to the surgeons via auditory signals (e.g. alarm or buzzer indicates when a pressure threshold has been exceeded) or through a visual display or light system.

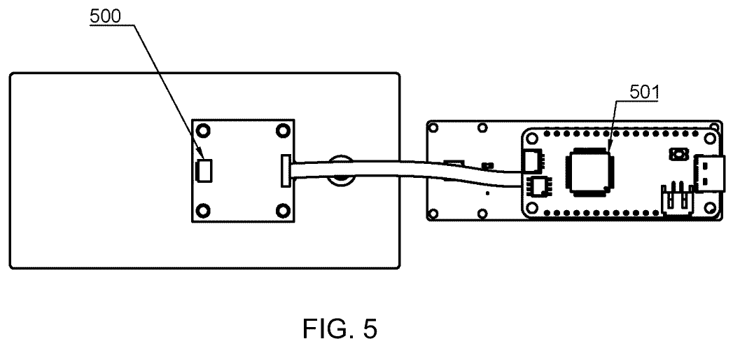

Referring to FIG. 5, one embodiment of the proposed device uses an ultra-miniature, ultra-low-power, optical data-acquisition system 500 (MAXM86161, Maxim Integrated) to obtain the reflected light signals. This package, which measures 2.9 mm×4.3 mm×1.4 mm, contains three LEDs and an on-board photodiode with multiplexed optical readout with 19-bit resolution (FIG. 2). The optical sensor module is controlled by a dedicated microcontroller system 501, which controls the collection of optical data and analysis of the signals to determine the contact pressure applied to the tissue during the retraction process.

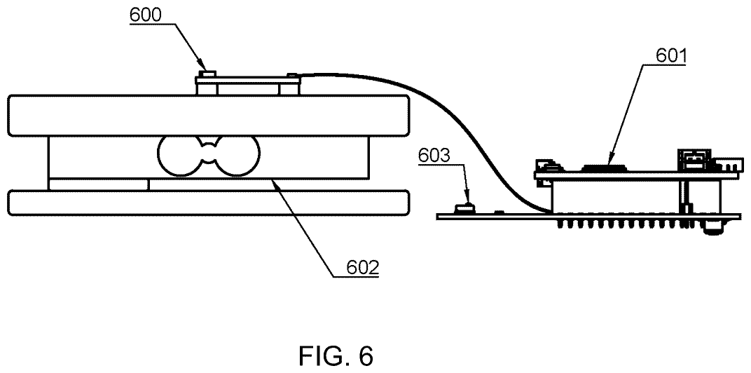

Referring to FIG. 6, in one embodiment an optical sensor module 600 is combined with a strain-gauge load cell 602, signal conditioner and amplifier 603 (HX711) to provide training data to an analysis algorithm, under conditions of known load. During training of the analysis algorithm, the applied pressure is measured simultaneously with the reflected optical signal while pressure is applied by tissue (e.g., a fingertip). The optical sensor module 600 (e.g. MAXM86161) and the load cell 602 are controlled by a rapid development platform 601 (MAX32630FTHR) that uses an ARM Cortex-M4F microprocessor. Custom software in the controller for the device allows for the computation of machine learning algorithms to determine the pressure calculations, as well as control the intensity, duration, and rate of the PPG light source pulses. It also allows for wired or wireless communications of the pressure values, as well as any signals or values of biological interest for the retracted tissue from the PPG signals.

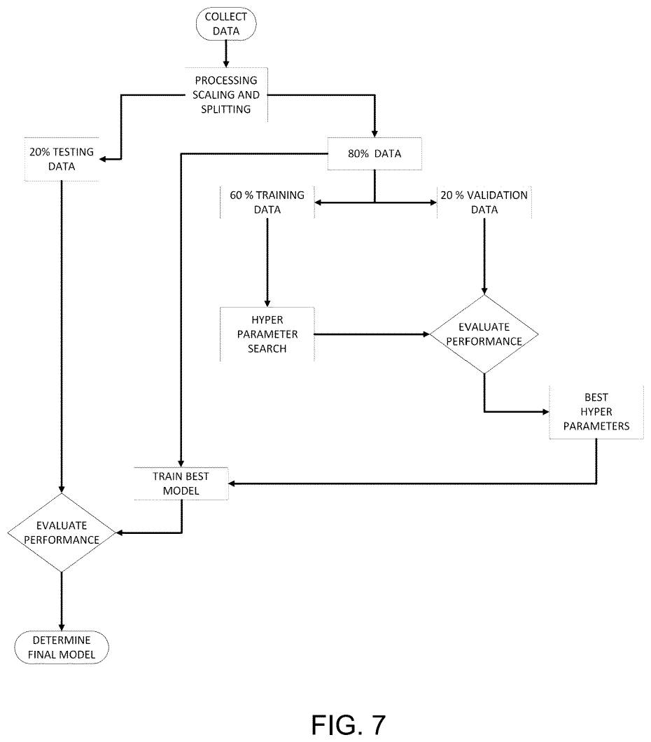

Referring to FIG. 7, we illustrate one method by which time-series analysis of the multi-spectral reflected optical signal can be performed by algorithms that predict the applied pressure or force. In this embodiment, training data (multispectral light signals acquired under conditions of known force) are used to train a machine-learning algorithm. Appropriate algorithms include but are not limited to: random-forest multiple decision trees, tree-based kth-nearest neighbour, and neural network regression machine-learning models, all of which can be trained to predict the relationship between the tissue pressure applied to the sensor and the resulting time-varying reflected light signal.

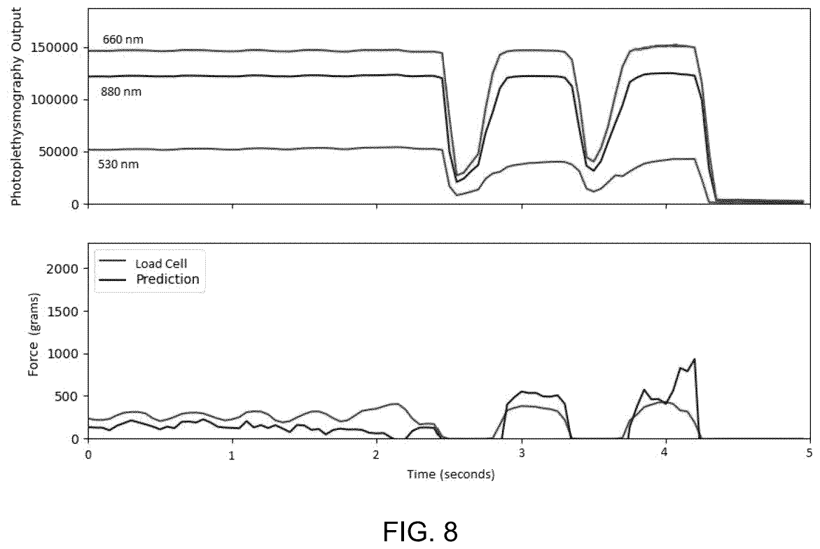

Referring to FIG. 8, we illustrate the results of one embodiment of the proposed system, where time-varying optical signals (at three wavelengths) are used to predict tissue force (fingertip) in real time.

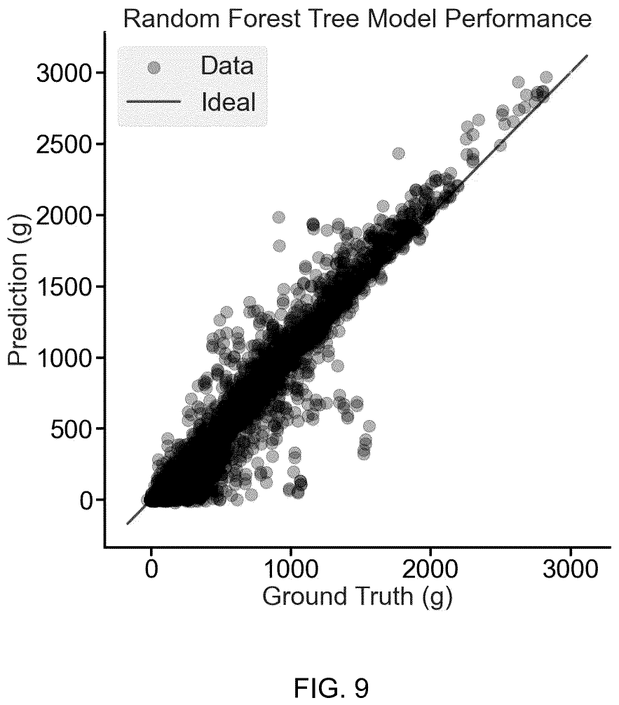

Referring to FIG. 9, we illustrate the results of testing of one analysis algorithm (random forest tree model) in predicting applied tissue force as a function of known applied force (in grams). The regression curve shows excellent agreement between pressure measured by the load cell (ground truth) and predicted by the machine-learning algorithm, based on reflected optical signals.

Further features and embodiments of the foregoing will be evident to persons of skill in the art. The inventors intend to cover all features, embodiments and sub-combinations thereof disclosed herein. The claims are to be construed as broadly as possible with reference to the specification as a whole.

What is claimed is:

1. A system for measuring pressure exerted on a tissue, the system comprising:
a medical device that exerts pressure on the tissue when the medical device is in contact with the tissue;
one or more optical sources coupled to the medical device, the one or more optical sources configured to direct an incident light signal to the tissue during operation of the medical device;
one or more optical sensors coupled to the medical device, the one or more optical sensors configured to receive and measure light-intensity, as a function of time, of a reflected and/or transmitted light signal that is reflected and/or transmitted from the tissue during the operation of the medical device;
a processor in electronic communication with the one or more optical sensors, the processor provided with machine-executable instructions to analyze the light-intensity as a function of the time to derive the pressure that the medical device exerts on the tissue at any given time; and,
one or more output devices for reporting information about the pressure the device exerts on the tissue at any given time.

2. The system of claim 1, wherein at least one of the one or more optical sources emits light at one or more wavelengths.

3. The system of claim 2, wherein at least one of the one or more optical sources emits continuous light or pulsed light.

4. The system of claim 2, wherein at least one the one or more optical sources comprises a light emitting diode or a laser.

5. The system of claim 2, wherein at least one of the one or more optical sensors detects light within a range of wavelengths that includes the one or more wavelengths of the emitted light.

6. The system of claim 1, wherein the one or more optical sources and the one or more optical sensors are integrated into a single module coupled to the medical device; or, wherein the one or more optical sources and the one or more optical sensors are independent components coupled to the medical device.

7. The system of claim 1, wherein the one or more optical sources and/or the one or more optical sensors are built into the medical device or are part of a flexible covering that covers the medical device; and, the one or more optical sources and the one or more optical sensors are coupled to the medical device.

8. The system of claim 1, wherein the one or more optical sources and/or the one or more optical sensors are directly in contact with the tissue, or the one or more optical sources and/or the one or more optical sensors are not directly in contact with the tissue and the light signals are guided by light guides.

9. The system of claim 8, wherein the light guides comprise fiber optic fibers.

10. The system of claim 1, wherein the medical device is a retractor configured to retract a portion of the tissue.

11. The system of claim 1, wherein measuring the light-intensity of the reflected and/or transmitted light signal comprises:
digitally measuring the reflected and/or transmitted light signal received by each of the one or more optical sensors;
communicating the light-intensity measurement to the processor;
storing a time series of the light-intensity measurements in an electronic memory; and,
reporting the light-intensity measurements using the one or more output devices.

12. The system of claim 1, wherein the processor is a single-board micro-controller provided with the machine executable instructions configured to execute an analysis program that: determines the exerted pressure from the light-intensity as a function of the time using an algorithm that has been trained to correlate the light-intensity as a function of the time to the exerted pressure at any given time; and, reporting the information about the pressure using the one or more output devices.

13. The system of claim 12, wherein the algorithm comprises decision trees, Kth nearest neighbours, neural networks, support vectors or XGBoost™.

14. The system of claim 12, wherein the information about the pressure comprises a warning, the analysis program determines when a pre-set pressure and/or time threshold has been exceeded, and the analysis program provides the warning as part of the information reported using the one or more output devices.

15. The system of claim 14, wherein the warning comprises a warning light, a tactile indication, a sound, or any combination thereof.

16. The system of claim 1, wherein the information comprises a report of when a specified pressure threshold is reached or a plot showing a history of the exerted pressure over time.

17. The system of claim 1, wherein the light-intensity of the reflected and/or transmitted light signal is used to measure vital signs of a patient, which are simultaneously reported with the pressure exerted on the tissue by the medical device using the one or more output devices.

18. The system of claim 17, wherein the vital signs comprise one or more of heart rate, blood oxygenation and blood pressure.

19. The system of claim 1, further comprising a temperature sensor for measuring temperature where the medical device contacts the tissue.

20. The system of claim 1, wherein the processor is external to the medical device and the system further comprises a communication port for coupling the one or more optical sensors to the processor.

21. The system of claim 20, wherein the communication port comprises a Universal Serial Bus (USB) port, an IEEE 1384 port, a serial port, a parallel port, a Personal Computer Memory Card International Association (PCMCIA) port, an Inter-Integrated Circuit (I2C) port, a Small Computer System Interface (SCSI) port, an optical port, a coaxial port, a Registered Jack 45 (RJ45) port and a Registered Jack 11 (RJ11) port, or a connector/connection for a mobile electronic device.

22. The system of claim 1, wherein:
a) the processor is external to the medical device and the system further comprises a wireless protocol for the electronic communication between the one or more optical sensors and the processor;
b) the system further comprises a power source for providing electrical power to the system, the power source comprising a battery; or,
both a) and b).
